(19) United States
(12) Patent Application Publication
(10) Pub. No.: US 2023/0357712 A1
(43) Pub. Date: Nov. 9, 2023

(54) A SODIUM ALGINATE, GELATIN, COLLAGEN AND FIBRIN (AGCF) BASED BIO-INK FOR THE BIOPRINTING OF A 3D BIOGEL-BASED TISSUE/STRUCTURE

(71) Applicant: THE STATE OF ISRAEL, MINISTRY OF AGRICULTURE& RURAL DEVELOPMENT, AGRICULTURAL, Rishon Lezion (IL)

(72) Inventors: Hinanit KOLTAI, Rishon LeZion (IL); Nurit SHALEV, Tirat Yehuda (IL)

(21) Appl. No.: 18/223,070

(22) Filed: Jul. 18, 2023

Related U.S. Application Data
(63) Continuation of application No. PCT/IL2022/050070, filed on Jan. 18, 2022.
(60) Provisional application No. 63/138,534, filed on Jan. 18, 2021.

(51) Int. Cl. C12N 5/00; C09D 105/04; C09D 189/04; B33Y 70/00; B33Y 80/00

(52) U.S. Cl. CPC C12N 5/0068; C09D 105/04; C09D 189/04; B33Y 70/00; B33Y 80/00; C12N 2513/00; C12N 2533/54; C12N 2533/70; C12N 2537/10; C12N 2533/50

(57) ABSTRACT

A bio-ink comprising: (a) at least one polysaccharide; (b) at least one structural protein; and (c) fibrinogen, wherein the polysaccharide is selected from a group consisting of Sodium alginate Chitosan, Hyaluronic acid, Gellan gum, Dextran, Agarose, Poly(ethylene glycol), Pluronic and Carrageenan and the structural protein is selected from a group consisting of Gelatin, Collagen Vitronectin, Laminins and Fibronectin.

### CROSS REFERENCE TO RELATED APPLICATIONS

This application is a bypass continuation of PCT Patent Application No. PCT/IL2022/050070 having International filing date of Jan. 18, 2022, which claims the benefit of priority of U.S. Provisional Patent Application No. 63/138,534, filed Jan. 18, 2021, the contents of which are all incorporated herein by reference in their entirety.

### FIELD OF THE INVENTION

The invention is in the field of bioprinting and tissue engineering.

### BACKGROUND OF THE INVENTION

Bioprinting has been developed as a new method of tissue engendering, specifically for the manufacturing of 3-dimensional structures. In some cases, the structures are used as a base for the biological material, such as cell. Recent methods for the 3-D printing, also known as additive manufacturing, have problems in accuracy and stability.

There is a long felt need for a composition and method for the manufacturing of tissue.

### SUMMARY

It is the object of the present invention to present a bio-ink comprising:
a. at least one polysaccharide;
b. at least one structural protein/scleroprotein/fibrous protein; and
c. fibrinogen;

wherein the polysaccharide is selected from a group consisting of Sodium alginate Chitosan, Hyaluronic acid, Gellan gum, Dextran, Agarose, Poly(ethylene glycol), Pluronic and Carrageenan and the structural protein/scleroprotein/fibrous protein is selected from a group consisting of Gelatin, Collagen Vitronectin, Laminins and Fibronectin.

It is another object of the present invention to present a bio-ink, as presented in any of the above, wherein the polysaccharide has undergone a preparation reaction.

It is another object of the present invention to present a bio-ink, as presented in any of the above, wherein the preparation is selected from a group consisting of oxidation and covalent derivatization.

It is another object of the present invention to present a bio-ink, as presented in any of the above, wherein the polysaccharide is characterized by at least one of the following:
a. having a size/molecular weight of 1.8-2.1×10⁵ g/mol;
a. soluble in water;
a. linear copolymer comprising β-1,4-d-mannuronic acid and α-1,4-1-guluronic acid;
b. comprising homopolymeric blocks of (1-4)-linked β-D-mannuronate (M) and C-5 epimer α-L-guluronate (G) residues;
c. binding and/or crosslinking with at least one 2⁺ ion;

It is another object of the present invention to present a bio-ink, as presented in any of the above, wherein the polysaccharide has undergone at least one preparation reaction.

It is another object of the present invention to present a bio-ink, as presented in any of the above, wherein the preparation is selected from a group consisting of heating, dissolving, esterification, oxidation, acetylation and amide formation reaction.

It is another object of the present invention to present a bio-ink, as presented in any of the above, wherein the preparation is characterized as a multicomponent reaction between at least one carboxylic acid functional group of the polysaccharides and at least one electrophile or nucleophiles.

It is another object of the present invention to present a bio-ink, as presented in any of the above, wherein the ratio of Gelatin to collagen is approximately 15:1 to 25:1.

It is another object of the present invention to present a bio-ink, as presented in any of the above, wherein the fibrinogen and the collagen are at a ratio of 3:1 to 1:3.

It is another object of the present invention to present a bio-ink, as presented in any of the above, additionally comprising cells.

It is another object of the present invention to present a bio-ink, as presented in any of the above, wherein the cells are characterized by being viable/alive.

It is another object of the present invention to present a bio-ink, as presented in any of the above, additionally comprising at least one crosslinking agent.

It is another object of the present invention to present a bio-ink, as presented in any of the above, wherein the crosslinking agent is selected from a group consisting of thrombin, calcium, copper, magnesium, manganese, strontium, barium, aluminum or a combination thereof.

It is another object of the present invention to present a bio-ink, as presented in any of the above, wherein the crosslinking agent is a serine protease.

It is another object of the present invention to present a bio-ink, as presented in any of the above, additionally comprising salts, acids, bases and buffer solutions

It is another object of the present invention to present a bio-ink, as presented in any of the above, wherein the salt is a calcium salt, a copper salt, magnesium salt, manganese salt, a strontium salt, a barium salt, an aluminum salt and a combination thereof.

It is another object of the present invention to present a bio-ink, as presented in any of the above, wherein the salt is a chloride salt or a sulfate.

It is another object of the present invention to present a bio-ink, as presented in any of the above, wherein the copper salt selected from a group consisting of copper (II) sulfate (CuSO₄), copper bromide (CuBr₂), copper fluoride (CuF₂), copper carbonate (CuCO₃), Copper nitrate (CuNO₃)₂, Copper oxide (CuO), Copper acetate Cu(OAc)₂ and Copper azide Cu(N₃)₂.

It is another object of the present invention to present a bio-ink, as presented in any of the above, characterized by at least one of the following:
a. viscosity in a range of 1-20000 Pa·S;
b. cell proliferation of ≥80%;
c. cell viability of ≥80%;
d. soluble in 0.05M EDTA+0.05M sodium citrate;
e. pH of 6.5-7.4;
f. boiling point of >100° C.;

It is the object of the present application to present a method of producing a bio-ink, comprising steps of:
a. preparing a solution of sodium alginate and gelatin;
b. preparing a solution of fibrinogen;
c. preparing a solution of collagen;
d. mixing the fibrinogen and collagen solutions;
e. adding cells to the fibrinogen and collagen solution;
f. mixing the cell, fibrinogen and collagen solution with the Sodium alginate and gelatin solution;

It is another object of the present invention to present a method of producing a bio-ink, as presented in any of the above, additionally comprising a step of heating the of Sodium alginate and gelatin solution to a temperature in the range of 50-80° c.

It is another object of the present invention to present a method of producing a bio-ink, as presented in any of the above, additionally comprising a step of sterilizing the solutions.

It is another object of the present invention to present a method of producing a bio-ink, as presented in any of the above, additionally comprising a step of filtering the solutions.

It is another object of the present invention to present a method of producing a bio-ink, as presented in any of the above, additionally comprising a step of centrifuge the solutions.

It is another object of the present invention to present a method of producing a bio-ink, as presented in any of the above, additionally comprising a step of filtering the solutions.

It is the object of the present invention to present a 3-d structure, comprising:
a. at least one polysaccharide;
b. at least one structural protein/scleroprotein/fibrous protein;
c. fibrinogen; and
d. at least one crosslinking agent
   wherein the stricture supports cell development and formation of secondary structures.

It is another object of the present invention to present a 3-d structure, as presented in any of the above, wherein the polysaccharide is selected from a group consisting of Sodium alginate Chitosan, Hyaluronic acid, Gellan gum, Dextran, Agarose, Poly(ethylene glycol), Pluronic and Carrageenan

It is another object of the present invention to present a 3-d structure, as presented in any of the above, wherein the structural protein/scleroprotein/fibrous protein is selected from a group consisting of Gelatin, Collagen Vitronectin, Laminins and Fibronectin.

It is another object of the present invention to present a 3-d structure, as presented in any of the above, comprising Gelatin and Collagen.

It is another object of the present invention to present a 3-d structure, as presented in any of the above, wherein the gelatin and the collagen are at a ratio of 15:1 to 25:1.

It is another object of the present invention to present a 3-d structure, as presented in any of the above, wherein the fibrin and the collagen are at a ratio of 1:3 to 3:1.

It is another object of the present invention to present a 3-d structure, as presented in any of the above, additionally comprising cells.

It is another object of the present invention to present a 3-d structure, as presented in any of the above, wherein the cells are characterized as being viable/alive.

It is another object of the present invention to present a 3-d structure, as presented in any of the above, characterized by at least one of the following:
a. viscosity in a range of 1-20000 Pa·S;
b. cell viability of ≥80%;
c. soluble in 0.05M EDTA+0.05M sodium citrate;
d. stability at a temperature range of 35 to 42° C.;

It is another object of the present invention to present a 3-d structure, as presented in any of the above, wherein the crosslinking agent is selected from a group consisting of thrombin, calcium, copper, magnesium, manganese, strontium, barium, aluminum and any combination thereof.

It is another object of the present invention to present a 3-d structure, as presented in any of the above, additionally comprising salts, acids, bases and buffer solutions and a combination thereof.

It is another object of the present invention to present a 3-d structure, as presented in any of the above, additionally comprising a calcium salt, a copper salt, a magnesium salt, a manganese salt, a strontium salt, a barium salt, an aluminum salt and a combination thereof.

It is another object of the present invention to present a 3-d structure, as presented in any of the above, wherein the salt is a chloride or a sulfate.

It is another object of the present invention to present a 3-d structure, as presented in any of the above, wherein the copper salt is selected from a group consisting of copper (II) sulfate (CuSO₄), copper bromide (CuBr₂), copper fluoride (CuF₂), copper carbonate (CuCO₃), Copper nitrate (CuNO₃)₂, Copper oxide (CuO), Copper acetate Cu(OAc)₂ and Copper azide Cu(N₃)₂.

It is the object of the present invention to present a method of printing a 3-D structure, comprising steps of:
a. obtaining the bio-ink of claim as presented in any of the above;
b. obtaining a printer and a temperature-controlled printer head;
c. preparing the printer head;
d. printing the bio-ink;
e. adding at least one crosslinking agent;
   wherein the crosslinking agent coagulates the bio-ink.

It is another object of the present invention to present a method of printing a structure, as presented in any of the above, additionally comprising a step of incubating the printed structure.

It is another object of the present invention to present a method of printing a structure, as presented in any of the above, wherein the crosslinking agent is selected from a group consisting of thrombin, calcium, copper, magnesium, manganese, strontium, barium, aluminum and a combination thereof.

It is another object of the present invention to present a method of printing a 3-D structure, as presented in any of the above, additionally comprising a step of adding salts, acids, bases and buffer solutions.

It is another object of the present invention to present a method of printing a 3-D structure, as presented in any of the above, additionally comprising a step of adding a calcium salt, a copper salt, magnesium salt, a manganese salt, a strontium salt, a barium salt, an aluminum salt or a combination thereof.

It is another object of the present invention to present a method of printing a 3-D structure, as presented in any of the above, wherein the salt is a chloride salt or a sulfate.

It is another object of the present invention to present a method of printing a 3-D structure, as presented in any of the above, wherein the calcium salt is selected from a group consisting of copper (II) sulfate (CuSO₄), copper bromide (CuBr₂), copper fluoride (CuF₂), copper carbonate (CuCO₃), Copper nitrate (CuNO₃)₂, Copper oxide (CuO), Copper acetate Cu(OAc)₂ and Copper azide Cu(N₃)₂.

It is another object of the present invention to present a method of printing a 3-D structure, as presented in any of the above, additionally comprising a step of sterilizing the solutions.

It is another object of the present invention to present a method of printing a 3-D structure, as presented in any of the above, additionally comprising a step of centrifuge the solutions.

It is another object of the present invention to present a method of printing a 3-D structure, as presented in any of the above, additionally comprising a step of filtering the solutions.

It is another object of the present invention to present a method of printing a 3-D structure, as presented in any of the above, wherein the printing is characterized by at least one of the following:
a. a temperature in a range of 20-25° C.;
b. a nozzle of 22G, 25G or 27G;
c. a speed of 1 to 3 mm/sec;
d. a pressure of 10 to 20 kPa;
e. an Infill density of <25%;

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the invention and are incorporated in and constitute a part of this specification, illustrate embodiments of the invention and together with the description serve to explain the principles of the invention wherein:

FIG. 1a presents (a) 24 well plate with rings printed as described above and contain GBM cells.

FIG. 1b presents a printed ring in 4× magnification.

FIG. 2 presents a confocal image of GBM cells (DAPI staining) 6 days after printing in AGCF 200 μm magnification.

FIG. 3 presents a confocal image of GBM cells (DAPI staining) 13 days after printing in AGCF. 200 μm magnification.

FIGS. 4a-4c present a confocal image of GBM cells (DAPI staining) 13 days after printing in AGCF: (FIG. 4a) 50 μm (FIG. 4b) 200 μm, (FIG. 4c) GBM sphere showed on day 13 at 10× magnification.

FIGS. 5a-5g present HCT 116 images:

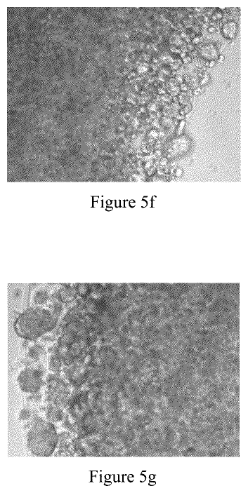

(FIG. 5a) 2^{nd} day-HCT 116. 4× magnification of Printed cylinder 5×1 structure,

(FIG. 5b) 2^{nd} day-HCT 116. 10× magnification of Printed cylinder 5×1 structure,

(FIG. 5c) 4^{nd} day-HCT 116. 4× magnification of Printed cylinder 5×1 structure,

(FIG. 5d) 4^{nd} day-HCT 116. 10× magnification of Printed cylinder 5×1 structure,

(FIG. 5e) 6^{nd} day-HCT 116. 4× magnification of Printed cylinder 5×1 structure,

(FIG. 5f) 6^{nd} day-HCT 116. 10× magnification of Printed cylinder 5×1 structure,

(FIG. 5g) 6^{nd} day-HCT 116. 10× magnification of Printed cylinder 5×1 structure

### DETAILED DESCRIPTION OF THE PREFERRED EMBODIMENT

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide compositions and methods.

In this application the term ‘ink’ or ‘bio-ink’ refers to printable biomaterials used in three dimensional (3D) bioprinting processes. In some embodiments, cells and other biologics are deposited in a spatially controlled pattern to fabricate living tissues and organs.

In this application the term ‘crosslinker’ refers to a compound (or compounds) that separately (or in combination) bond one polymer chain (or one part of a chain) to another polymer chain (or another section of the same part of a chain).

In this application the terms ‘crosslinking agent’ or ‘crosslinking factor’ refers to a compound (or compounds) that separately (or in combination) create a bond between one polymer chain (or one part of a chain) to another polymer chain (or another section of the same part of a chain).

Unless otherwise stated, with reference to numerical quantities, the term “about” refers to a tolerance of ±25% of the stated nominal value.

Unless otherwise stated, all numerical ranges are inclusive of the stated limits of the range.

### DETAILED DESCRIPTION OF THE INVENTION

Bioprinting systems commonly have three major segments: hardware (printer), bio-ink and printing template.

Three main types of printer technologies are use in bioprinting: inkjet, laser-assisted, and extrusion printers. Inkjet printers are mainly used for fast printing and large-scale products. One type of inkjet printer, called drop-on-demand inkjet printer, prints materials in exact amounts, minimizing cost and waste. Laser-assisted printing are commonly used to provide high-resolution printing.

Extrusion printers print cells layer-by-layer to create 3D constructs.

The bio-ink serves to create a 3-D structure, that serves as a basis for the growth delivery medium for (living) cells, that behaves much like a liquid, enabling the printing of the desired shape. The components of the ink of the present invention can be divided into 2 main groups according to the function:
Structural components, that can be sub-divided by chemical classification:
   Structural polysaccharides:
      Sodium alginate, also called alginic acid, is a polysaccharide distributed widely in the cell walls of brown algae that is hydrophilic and forms a viscous gum when hydrated. Sodium alginate is a linear copolymer with homopolymeric blocks of (1-4)-linked β-D-mannuronate (M) and its C-5 epimer α-L-guluronate (G) residues. The M and G blocks may appear as alternates or consecutive.
      Chitosan, a natural cationic linear copolymer of β-(1-4) linked 2-acetamino-2-deoxy-d-glucopyranose and 2-amino-2deoxy-d-glucopyranose, is a deacetylated form of chitin derived from shells of crustaceans.
      Hyaluronic acid (HLA), a linear non-sulfated glycosaminoglycan (GAG), is a polysaccharide with the repeating disaccharide, β-1,4-d-glucuronic acid, β-1,3-N-acetyl-d-glucosamine. HLA is a major ECM component of cartilage and ubiquitously find in almost all connective tissues. In some cases, the HLA is pre-treated before printing to improve stability and it's mechanical properties.
      Gellan gum is a hydrophilic and high-molecular weight anionic polysaccharide produced by bacteria. In some cases, gellan gum is chemically modified to lower the gelation temperature of compositions comprising gelation temperature.
      Dextran, a natural water-soluble polysaccharide. In some cases, the Dextran is modified by a chemical process, such as oxidation.
      Agarose, is a polysaccharide extracted from marine algae and seaweed.
      Carrageenan, a family of linear sulfated polysaccharides, which are extracted from the red seaweeds. The main chain consists of repeating units of β-D-galactose and α-D-galactose.
      Synthetic polysaccharides, such as Poly(ethylene glycol) (PEG) and Pluronic (a type of poloxamer). In some cases, the polymers are treated to digest or degrade the cross-linking bonds.
   Structural proteins (also known as Scleroproteins or fibrous proteins) are commonly constructed by elongated or fibrous polypeptide chains which form filamentous and sheet like structure. Scleroproteins typically have low solubility in water. In some embodiments, the structural proteins also promote cell attachment and proliferation.
      Collagen is the main structural protein in the extracellular matrix in the various connective tissues in the body and consists of amino acids bound together to form a triple helix of elongated fibril (known as a collagen helix). Collagen is the most abundant protein in mammals, making up from 25% to 35% of the whole-body protein content. Collagen forms a triple helix of elongated fibril, known as a collagen helix. The hardness or rigidity of the collagen tissue depending upon the degree of mineralization, ranging from rigid (such as bones) to compliant (such as a tendon) or as a gradient covering the range from rigid to compliant (such as cartilage). The Collagen could be of type I-V and from various (mammalian) sources, such as calf skin.
      Gelatin (also referred to as gelatine, hydrolyzed collagen, collagen hydrolysate, gelatine hydrolysate and hydrolyzed gelatine) is a translucent, colorless and flavorless material often used as a food ingredient. Gelatin is derived from collagen that has been partially purified and hydrolyzed. Collagen hydrolysis is performed by one of three different methods: acid-, alkali-, and enzymatic hydrolysis. Gelatin is brittle when dry and gummy when moist. Gelatin forms thermally reversible gels with water and can form stable foams.
      Both collagen and gelatin are 98-99% protein by dry weight, with the amino acid content being 24-25% proline and/or hydroxyproline, 20-21% glycine, 10-11% glutamic acid, 8% arginine, 8-9% alanine and 28% other amino acids.
      Vitronectin (VTN or VN) is a glycoprotein of the hemopexin family which is abundantly found in serum, the extracellular matrix and bone
      Laminins are high-molecular weight (˜400 to ˜900 kDa) glycoproteins of the extracellular matrix. They are a major component of the basal lamina (one of the layers of the basement membrane), a protein network foundation for most cells and organs. Laminins are heterotrimeric proteins that contain an α-chain, a β-chain, and a γ-chain (often found in five, four, and three genetic variants, respectively). The trimeric proteins intersect to form a cross-like structure that can bind to other cell membrane and extracellular matrix molecules:
         The three shorter arms cand bind to other laminin molecules, forming larger structures (such as sheets).
         The long arm binds to cells, anchoring the structure to the membrane.
      Fibronectin is a high-molecular weight (˜440 kDa) glycoprotein component of the extracellular matrix that binds to membrane-spanning receptor proteins called integrins, in addition to other extracellular matrix proteins such as collagen, fibrin, and heparan sulfateproteoglycans (e.g. syndecans). Two types of fibronectin are present in vertebrates: soluble plasma fibronectin (a major protein component of blood plasma) and insoluble cellular fibronectin (a major component of the extracellular matrix).
Functional components, that can enable the ink-to form a stable 3-D structure or to enable cell-colonization:
   A crosslinker (binding/crosslinking/clotting protein):
      Fibrinogen is a large, fibrous, non-globular and soluble glycoprotein that is involved in blood clotting, as thrombin converts it into insoluble fibrin molecule, in the presence of Ca²⁺ via intermolecular interactions. Thrombin converts fibrinogen to fibrin and then to a fibrin-based blood clot. Fibrin clots function primarily to occlude blood vessels to stop bleeding The fibrinogen protein is a 45 nm hexameric arrangement of peptide chains (alpha, beta, and gamma) connected by disulfide bonds, with a molecular weight of approximately 340 kDa. The fibrinogen protein consists of two outer D domains and a central E domain and is encoded by chromosome 4. Fibrin can form a fibrin scaffold as a network of protein that holds together and supports a variety of living tissues. It is produced naturally by the body after injury, but also can be engineered as a tissue substitute to speed healing. The scaffold consists of naturally occurring biomaterials composed of a cross-linked fibrin network and has a broad use in biomedical applications.
      Fibrinogen can further promote cell attachment and proliferation.

In some embodiments, the components can belong to more that one classification.
Other:
   Crosslinking agent or factor, referring to a compound (or compounds) that induces bonding (or crosslinking) via at least one component of the ink. The ink may include more than one crosslinking agent, with each agent creating binds to the same or different components. The bond can be covalent on ionic.
   In some embodiments, the crosslinker is a coagulating agent:
      Thrombine is a serine protease that converts soluble fibrinogen into insoluble strands of fibrin. Thrombine attacks the N-terminus of the Act and Bβ chains in fibrinogen to form individual fibrin strands plus two small polypeptides, fibrinopeptides A and B, derived from these respective chains. The individual fibrin strands aggregate to form a three-dimensional gel-like structure by polymerizing and crosslinking with other fibrin stands. Fibrin regulates the aggregation by possessing three low affinity binding sites (two in fibrin's E domain; one in the D domain) for thrombin; this binding sequesters thrombin from attacking fibrinogen.
      The processes happen in the presence of 2⁺ ions (such as calcium).
      Cerastocytin, a thrombin-like serine protease, such as found in snake venom.
      Calcium (Ca²⁺), often added as a salt (such as Calcium chloride, CaCl₂)) is necessary for blood clotting and is also referred to as co-factor IV. Calcium binds to Thrombin and fibrinogen. It has been proposed that Ca′ forms a Ca⁺-dependent dimer that catalyzes fibrinogen proteolysis by thrombin. 2⁺ Ions, such as calcium, magnesium, manganese, strontium and copper, that can also reversibly crosslink polysaccharides, such as Sodium alginate, in a rapid manor (crosslinking of Sodium alginate by calcium can be completed in 2-5 minutes).

The bio-ink may also comprise additional minor components, such as:
Solvents, such as water-based buffers (such as PBS and HEPES) and glycerol-water solutions. In many cases the buffer is effective at pH 6.8 to 8.2 (pKa 7.55).
Cells: the cells colonize the printed 3-D structure. In some embodiments, the cells are harvested from the subject/patent before the printing. In some embodiments, the cells are harvested from a donor.
In some embodiments, the cells are subjected to a procedure before being adding to the printed structure, such as incubation etc.
Additional ingredients:
   Salts:
      Calcium (Ca⁺), added as Calcium chloride (CaCl₂)), is necessary for blood clotting, Calcium is also referred to as co-factor IV. Calcium binds to Thrombin and fibrinogen. It has been proposed that Ca²⁺ forms a Ca²⁺-dependent dimer that catalyzes fibrinogen proteolysis by thrombin.
      Divalent metal ions such as Mg⁺, Mn²⁺Al²⁺ and Sr⁺, often added as Chloride salts, has been show to interact with Thrombin and fibrinogen to facilitate the crosslinking process (MgCl₂, MnCl₂ and SrCl₂).
   Acids (such as Acetic Acid and Sulphoric acid) and bases (such as NaOH), to be used as co-solvents and for regulating pH of the solution. Most acids and bases are used in aqueous solutions. In many embodiments, the solutions are in concentrations of <0.5M.
   Additional (minor) components, that can be osmolytes (such as D-mannitol) or stabilizers (such as TWEEN) can be added. Some of these components, such as D-mannitol, assist in maintaining the bioink's osmolarity, and therefore the for the compatibility of mammalian cells in the bioink,

A general method for preparation of the bio-ink of the present invention:
a. Preparing a solution of Sodium alginate and gelatin;
b. Heating solution (in a range of 50-80° C.);
c. Preparing a solution of fibrinogen;
d. Preparing a solution of collagen;
e. Mixing fibrinogen and collagen solutions
f. Adding cells to fibrinogen-collagen solution
g. Mixing cell, fibrinogen and collagen solution with Sodium alginate and gelatin solution

The final bio-ink product comprises the ingredients in a ratio of:

TABLE 1
composition of bio-ink

| | | |
|---|---|---|
| Ingredient | % of ink components | Content Range |

| | | | | |
|---|---|---|---|---|
| Sodium alginate | 23 | (20-30) | 20-25 | mg/ml |
| Gelatin | 60 | (55-65) | 40 to 50 | mg/ml |
| Fibrinogen | 10 | 7-12) | 40 to 60 | mg/ml |
| Collagen | 2 | (1-3) | 2 to 3 | mg/ml |

| Solvents | % of solvent | |
|---|---|---|
| PBS | 90 | 800-900 ul/ml |
| Glycerol | 10 | 100 to 200 ul/ml |
| Acetic Acid | 0.1 | 10 to 20 ul/ml |

General method for printing a structure, using the bio-ink of the present invention:
a. Preparing the bio-ink (according to the method disclosed above)
b. Setting printing head and template temperature
c. Preparing the crosslinking solution
d. Printing, conducted in conditions suitable for cell viability (temperature range of 20 to 40° C. and pressure of 10 to 20 kPa)
e. Adding crosslinker solution to printed structure
f. Washing structure in a cell medium, such as Minimal Essential Medium (MEM) or other (synthetic) cell culture medium.
g. Incubate structure in a CO₂ incubator with in MEM.

The final product could be considered as a Hydrogel, a class of crosslinked polymeric substances capable of absorbing and retaining large quantities of water.

The final printed product comprises the ingredients in a ratio of:

TABLE 2
composition of printed structure

| | % of Ink | | Content Range | |
|---|---|---|---|---|
| | ingredients | | (per 0.1 ml) | |
| Ingredient | | | | |
| Sodium alginate | 23 | (20-25%) | 2-2.5 | mg |
| Gelatin | 60 | (55-65%) | 4 to 5 | mg |
| Fibrinogen | 10 | (8-12%) | 4 to 6 | mg |
| Collagen | 2 | (1-3%) | 0.2 to 0.3 | mg |

| | | | |
|---|---|---|---|
| Cells | 5 | (2-10%) | 100,000 to 500,000 |

| Additional components | | | |
|---|---|---|---|
| Solvents (PBS, glycerol) | | 60-100 | ul |
| Acids (Acetic Acid) | Trace | 3-5 | ul |
| | amount (0.01%) | | |
| Crosslinking agent | 10 | 50 | ul |
| Salts (CaCl₂) | 0.1 | | |

The MEM that can be used to maintain cells in tissue culture and comprises salts (such as calcium chloride, potassium chloride, magnesium sulfate, sodium chloride, sodium phosphate and sodium bicarbonate), glucose, amino acids, and vitamins (such as thiamine (vitamin B₁), riboflavin (vitamin B₂), nicotinamide (vitamin B₃), pantothenic acid (vitamin B₅), pyrodoxine (vitamin B₆), folic acid (vitamin B₉), choline, and myo-inositol (originally known as vitamin B₈). In some embodiments, the MEM is Dulbecco's modified Eagle's medium (DMEM).

A more detailed method for the production of an ink would be:
1) Sterilizing the Sodium alginate and gelatin, using UV exposure for 20 min
2) Wash the magnetic bead with 100% ethanol for 30 min and autoclave the magnetic beads, Scintillation Vials, 3 mL cartridges with tip caps, Nozzles, Female/female Luer lock adapter.
3) Mix the 9 ml PBS and 1 ml glycerol and filter through 0.45 uM
4) Dissolve Sodium alginate (20 mg) and gelatin (40 mg) in 1 ml PBS:glycerol (9:1), and pin sterile scintillation vial having magnetic bead.
5) Heat the solution at 70° C. for 40 min with constant stirring.
6) Transfer the solution/mixture/gel to cartridges.
7) Centrifuge the cartridge (4000 rpm for 10 min) to remove air bubbles. The gel can be stored at 4° C.
8) Dissolve the fibrinogen (50 mg/ml) in PBS by shaking for 30 min at 30° C.
9) Dissolve the collagen (1 mg/ml) in a 0.1M acetic acid solution.
10) Neutralized the collagen solution using 0.1N NaOH 2.2 mg/ml to a pH of 6-7.
11) Mix the fibrinogen and collagen in 1:1 ratio and filter through 0.45 uM. The solution of 300 ul comprises 7.5 mg Fibrinogen and 0.3 mg collagen.
12) Harvest the cells and generate the cell pellet having appropriate number of cells (approximately 2 million to 8 million cells).
13) Dissolve the cell pellet in 300 ul of fibrinogen and collagen mixture (prepared in step 10) and transferred to 3 ml syringe (approximately 2 million to 8 million cells).
14) 1 ml Sodium alginate and gelatin solution (step 7) in a sterile cartridge (3 ml) is mixed with 0.3 ml of the cells, fibrinogen and collagen solution.
15) Set the temperate in a temperature-controlled print head and print bed.
16) Prepare template. In some embodiments, the template is a cylinder 5×1 mm·stl.
17) Printing: Bio printing procedure: temperature 20-25° C., Nozzle-25G, Speed:1 mm/sec, pressure-10-20 kPa, Infill density-0%, 3D model-cylinder 5×1.
18) Preparing crosslinking solution: 990 ul CaCl₂) and 10 ul thrombine are dissolved in PBS. The solution can be stored at ˜−20° C.
19) Add crosslink solution (step 17) to the printed structure (step 16) and incubate for 2-5 min.
20) The printed structure is washed twice with DMEM Complete media (2×500 ul).
21) Add 500 ul DMEM Complete media and transferred to CO₂ incubator. In some embodiments, the incubation is for a period of approximately 14 days

### EXAMPLES

Material and Methods: Cell Culture: GBM, HCT116, HaCaT

HCT116: HCT116 (ATCC CCL-247) colon cells were grown at 37° C. in a humidified 5% CO₂-95% air atmosphere. Cells were maintained in McCoy's 5a Modified Medium having 10% fetal bovine serum and 1% Penstrep. Cells were grown in 250 ml cell culture flask by giving 10 ml complete media twice a weekly. On reaching 80-90% confluence, cells are detached using 1.5 ml Trypsin EDTA solution (0.25% trypsin and 0.05% EDTA) in 5 min, added 10 ml McCoys complete media. For Bioprinting 8×10⁶ cells were mixed with 300 ul of collagen: fibrinogen.

GBM: A172 (ATCC® CRL-1620™) glioblastoma cells were grown at 37° C. in a humidified 5% CO₂-95% air atmosphere in T-75 flasks. Cells were maintained in complete DMEM Medium (with 10% FBS, 5 ml pen-strep, 5 ml L-glutamine, 100 ul plasmocin).

The GBM 3d structures printed into 24 wells plate in complete DMEM media at concentration of 8 million cells per mL. After 24 hours incubation, the medium replaced to serum-free DMEM/F-12 (5004, per well).

Procedure for Preparation of ECM (1.8% Sodium Alginate+Gelatin+Collagen+Fibrinogen [AGCF] BioGel)

1) Sterilization of Sodium alginate and gelatin using UV exposure for 20 min

2) Wash the magnetic bead with 100% ethanol for 30 min and autoclave the magnetic beads, Scintillation Vials, 3 mL cartridges with tip caps, Nozzels, Female/female Luer lock adapter.

3) 1.8% Sodium alginate=Dissolve the 39.6 mg Sodium alginate and 90 mg gelatin in 2 ml solvent (mix 1.8 ml pbs with 0.2 ml glycerol and filter through 0.45 uM) in sterile scintillation vial having magnetic bead.

4) Heat the solution at 70° C. for 40 min with constant stirring.

5) Transfer the gel to 3 mL cartridges with end and tip caps.

6) Centrifuge the cartridge at 4000 rpm for 10 min to remove air bubbles and Store at 4° C.

7) Dissolve the 50 mg/ml fibrinogen in PBS in shaker for 30 min at 30° C.

8) Dissolve the 2.2 mg/ml collagen in 0.1M acetic acid and neutralized the solution using 1N NaoH.

9) Mix the fibrinogen and collagen in 1:1 ratio and filter through 0.45 uM

10) Preparation of crosslinking agent: mix 990 ul CaCl₂ and 10 ul thrombine and keep cooled (in ice).

11) Harvest the cells and generate the cell pellet having appropriate number of cells (as indicated in results).

12) Dissolve the cell pellet in 300 ul fibrinogen and collagen mixture (prepared in step 9) and transferred to 3 ml syringe.

13) Take 1 ml of 1.8% Sodium alginate in sterile 3 ml cartridges and mix thoroughly using 3 ml syringe having cells+fibrinogen+collagen.

14) Set the print head and print bed to 20° C.

Bio Printing Procedure: Nozzle-25G, Speed-1 mm/Sec, Pressure-20 kPa, Infill Density-0%, 3D Model-Cylinder 5×1

15) Add crosslink agent (step 10) and incubate for 5-10 min, wash the structure with DMEM Complete media (2times×500 ul)

16) Add 500 ul DMEM Complete media and transferred to CO₂ incubator

17) After 3 days, aspirated media and added serum free DMEM media, changed media for every 3 days

Confocal image was taken at day 1 and then the following days to determine cell proliferation and organization. Cells were stained with DAPI (Fluoroshield with DAPI-F6057) and cell nuclei were detected.

DAPI staining:
1) Transfer carefully the 3D structure from 24 well plate to confocal plate 2) Wash the structure with 500 ul PBS for 2 times
3) Add Dapi 2-3 drops to cover whole structure under dark
4) Incubate in CO₂ incubator for 45 min
5) To Confocal Microscope

Digestion Procedure:

1) Dissolve 1.47 gm of trisodium citrate dehydrate in 10 ml 0.5M EDTA and filter through 0.22 uM (tube 1)

2) Dissolve 0.3 gm sodium chloride in 10 ml water (tube 2) and transfer 3 ml from tube 1 to 7 ml water and filter through 0.22 uM (tube 3)

3) Digestion solution 0.05M sodium citrate+0.05M EDTA: Take 9 ml solution from tube 3- and 1-ml solution from tube 1, mix thoroughly and store in ice basket

4) Transfer the 3D str. To 1.5 ml vial and wash with PBS (500 ul×2 times) 5) Add 200 ul digestion solution (step 3) and mix thoroughly using tip (10 to 20 times) until the str. Disappear completely

6) Add 200 ul DMEM complete media and centrifuge at 1300 rpm/3 min

7) Wash the pellate with 500 ul PBS-2 times

8) Add 500 ul DMEM complete media and transfer the cell to 24 well plate 9) Next Day Aspirate the media and add 500 ul media to the attached cells

Results:

1. Glioblastoma (GBM)

1.1 Microscopic Observation

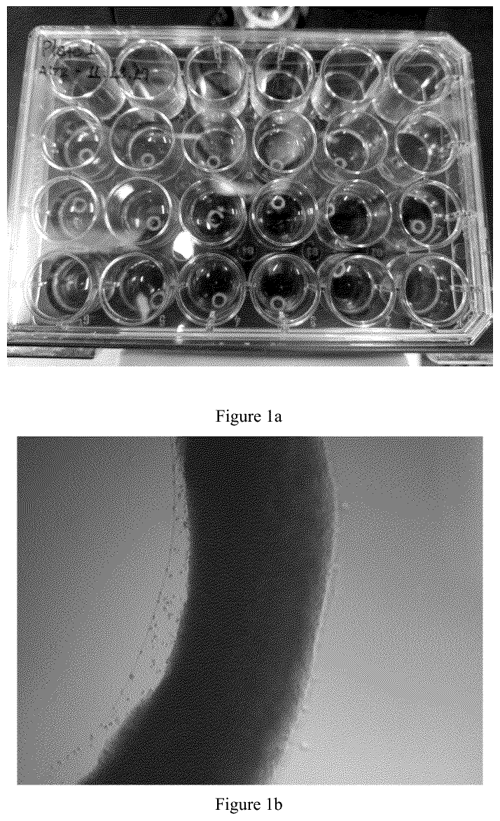

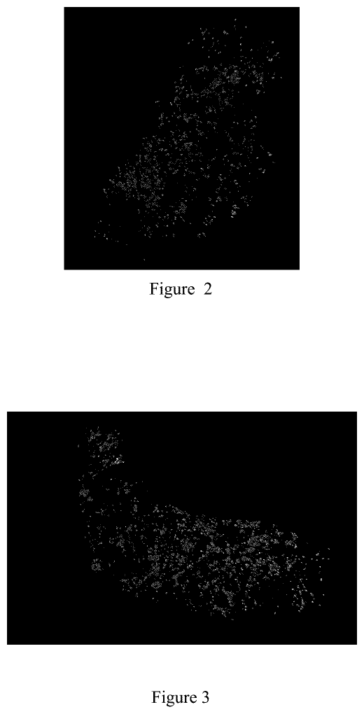

Reference is made to FIG. 1, demonstrating the pelleting and printing of 8 million cells in a circle structure (as described above).

The cells were stained with DAPI and observed, as optical sections, by confocal. Image acquisition was done Leica SP8 laser scanning microscope (Leica, Wetzlar, Germany), equipped with a solid-state laser with 405 nm light, HC PL APO CS 10×/0.40 objective (Leica, Wetzlar, Germany) and Leica Application Suite X software (LASX, Leica, Wetzlar, Germany). DAPI emission signals were detected with PMT detector in ranges of 415-490 nm high.

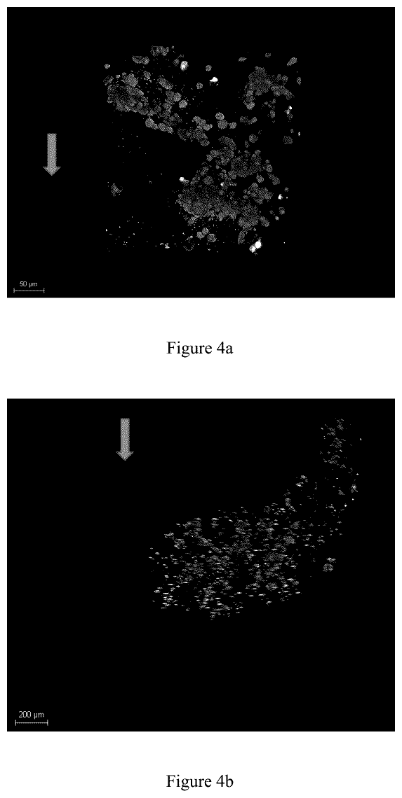

Reference is made to FIG. 2, demonstrating the homogenously spread of the cells through the printed structure at day 6.

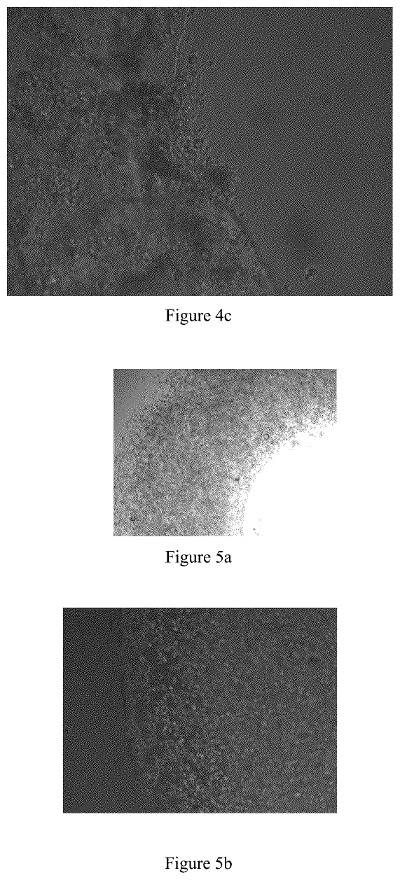

Reference is made to FIG. 3, demonstrating the accumulation of cells at day 13, forming nets of accumulated cells.

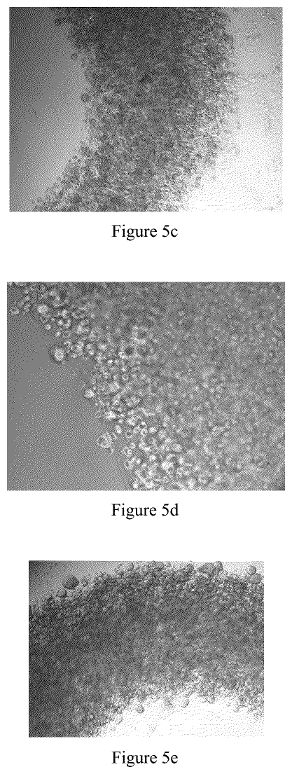

Reference is made to FIG. 4, demonstrating the presence of new secondary structures that contain relatively small and packed cells, formed in places of accumulated using a progenitor cell. The secondary structures (FIG. 4, arrow) are usually referred to as colonies or spheres. These development processes are similar to those documented in 2D tissue cultures (Lopez-Bertoni H, Lal B, Li A, Caplan M, Guerrero-Cazares H, Eberhart CO, Quinones-Hinojosa A, Glas M, Scheffler B, Laterra J, Li Y. DNMT-dependent suppression of microRNA regulates the induction of GBM tumor-propagating phenotype by Oct4 and Sox2. Oncogene. 2015 July; 34(30):3994-4004.) These spheres (neurospheres) are sub-populations of multipotent stem-like cells and efficiently propagate tumors in xenograft models, reflecting their self-renewing and tumor-propagating capacity. Evidences suggest that these stem-like cells have important role in maintaining tumor growth, therapeutic resistance and tumor recurrence (Lopez-Bertoni et al., 2015).

Our results suggest that GBM cells in 3D structures of AGCF develop similarly to GBM cells in 2D culture plates.

2. Colorectal Cancer (CRC)

8 million cells were pelleted and printed in a circle structure as described above (FIG. 1). Bright field images of the printer structure are below (FIG. 5).

1-50. (canceled)

51. A bio-ink comprising:
a. at least one polysaccharide;
b. at least one structural protein; and
c. fibrinogen;
wherein said polysaccharide is selected from a group consisting of sodium alginate, chitosan, hyaluronic acid, gellan gum, dextran, agarose, poly(ethylene glycol), pluronic and carrageenan and said structural protein is selected from a group consisting of gelatin, collagen vitronectin, laminins and fibronectin.

52. The bio-ink of claim 51, wherein said polysaccharide has undergone a preparation reaction, further wherein at least one of the following is true:
a. said preparation reaction is selected from a group consisting of oxidation and covalent derivatization;
b. said preparation is selected from a group consisting of heating, dissolving, esterification, oxidation, acetylation and amide formation reaction; and
c. said preparation is characterized as a multicomponent reaction between at least one carboxylic acid functional group of said polysaccharides and at least one electrophile or nucleophiles.

53. The bio-ink of claim 51, wherein said polysaccharide is characterized by at least one of the following:
a. Having a size/molecular weight of 1.8-2.1×10⁵ g/mol;
b. Soluble in water;
c. linear copolymer comprising β-1,4-d-mannuronic acid and α-1,4-1-guluronic acid;
d. comprising homopolymeric blocks of (1-4)-linked β-D-mannuronate (M) and C-5 epimer α-L-guluronate (G) residues; and
e. binding and/or crosslinking with at least one 2⁺ ion.

54. The bio-ink of claim 51, wherein at least one of the following is true:
a. the ratio of said gelatin to said collagen is approximately 15:1 to 25:1; and
b. the ratio of said fibrinogen to said collagen are at a ratio of 3:1 to 1:3.

55. The bio-ink of claim 51, additionally comprising cells.

56. The bio-ink of claim 55, wherein said cells are characterized by being viable/alive.

57. The bio-ink of claim 51, additionally comprising at least one crosslinking agent, wherein at least one of the following is true:
a. said crosslinking agent is selected from a group consisting of thrombin, calcium, copper, magnesium, manganese, strontium, barium, aluminum or a combination thereof; and
b. said crosslinking agent is a serine protease.

58. The bio-ink of claim 51, additionally comprising salts, acids, bases and buffer solutions, further wherein one of the following is true:
a. said salt is a calcium salt, a copper salt, magnesium salt, manganese salt, a strontium salt, a barium salt, an aluminum salt and a combination thereof; and
b. said salt is a chloride salt or a sulfate.

59. The bio-ink of claim 58, wherein said copper salt is selected from a group consisting of copper (II) sulfate (CuSO₄), copper bromide (CuBr₂), copper fluoride (CuF₂), copper carbonate (CuCO₃), Copper nitrate (CuNO₃)₂, Copper oxide (CuO), Copper acetate Cu(OAc)₂ and Copper azide Cu(N₃)₂.

60. The bio-ink of claim 56, characterized by at least one of the following:
a. viscosity in a range of 1-20000 Pa·S;
b. cell proliferation of ≥80%;
c. cell viability of ≥80%;
d. soluble in 0.05M EDTA+0.05M sodium citrate;
e. pH of 6.5-7.4; and
f. a boiling point of >100° C.

61. A method of producing a bio-ink, comprising steps of:
a. preparing a solution of sodium alginate and gelatin;
b. preparing a solution of fibrinogen;
c. preparing a solution of collagen;
d. mixing said fibrinogen and collagen solutions;
e. adding cells to said fibrinogen and collagen solution; and
f. mixing said cell, fibrinogen and collagen solution with said sodium alginate and gelatin solution.

62. The method of claim 61, additionally comprising at least one of the following steps:
a. heating said of sodium alginate and gelatin solution to a temperature in the range of 50-80° C.;
b. sterilizing said solutions;
c. filtering said solutions;
d. centrifuging said solutions; and
e. filtering said solutions.

63. A 3D structure, comprising:
a. at least one polysaccharide;
b. at least one structural protein/scleroprotein/fibrous protein;
c. fibrinogen; and
d. at least one crosslinking agent,
wherein said stricture supports cell development and formation of secondary structures.

64. The 3D structure of claim 63, wherein said polysaccharide is selected from a group consisting of sodium alginate, chitosan, hyaluronic acid, gellan gum, dextran, agarose, poly(ethylene glycol), pluronic and carrageenan.

65. The 3D structure of claim 63, wherein said structural protein/scleroprotein/fibrous protein is selected from a group consisting of gelatin, collagen, vitronectin, laminins and fibronectin.

66. The 3D structure of claim 63, additionally comprising viable/alive cells.

67. The 3D structure of claim 63, additionally comprising at least one crosslinking agent, wherein at least one of the following is true:
a. said crosslinking agent is selected from a group consisting of thrombin, calcium, copper, magnesium, manganese, strontium, barium, aluminum or a combination thereof; and
b. said crosslinking agent is a serine protease.

68. The 3D structure of claim 63, additionally comprising salts, acids, bases and buffer solutions, further wherein one of the following is true:
a. said salt is a calcium salt, a copper salt, magnesium salt, manganese salt, a strontium salt, a barium salt, an aluminum salt and a combination thereof; and
b. said salt is a chloride salt or a sulfate.

69. The 3D structure of claim 63, wherein said copper salt is selected from a group consisting of copper (II) sulfate (CuSO₄), copper bromide (CuBr₂), copper fluoride (CuF₂), copper carbonate (CuCO₃), Copper nitrate (CuNO₃)₂, Copper oxide (CuO), Copper acetate Cu(OAc)₂ and Copper azide Cu(N₃)₂.

70. The 3D structure of claim 66, characterized by at least one of the following:
a. viscosity in a range of 1-20000 Pa·S;
b. cell viability of ≥80%;
c. soluble in 0.05M EDTA+0.05M sodium citrate; and
d. stability at a temperature range of 35-42° C.
